(19) United States
(12) Patent Application Publication
(10) Pub. No.: US 2003/0236633 A1
(43) Pub. Date: Dec. 25, 2003

(54) Methods for oligonucleotide probe design

(75) Inventors: Rui Mei, Santa Clara, CA (US); Teresa A. Webster, Loma Mar, CA (US); Mike Mittmann, Palo Alto, CA (US); Mei-Mei Shen, Cupertino, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/310,013

(22) Filed: Dec. 4, 2002

Related U.S. Application Data
(63) Continuation of application No. 10/017,034, filed on Dec. 14, 2001.
(63) Continuation of application No. 09/718,295, filed on Nov. 21, 2000.
(60) Provisional application No. 60/335,012, filed on Oct. 25, 2001.

(51) Int. Cl. G06F 19/00; G01N 33/48; G01N 33/50

(52) U.S. Cl. 702/20

(57) ABSTRACT

In one embodiment of the invention, methods are provided for oligonucleotide probe arrays for gene expression monitoring.

### RELATED APPLICATIONS

This application claims the priority of U.S. patent application Ser. Nos. 09/718,295, 10/017,034, and U.S. Provisional application No. 60/335,012 and U.S. patent application Ser. No. ______, attorney docket number 3359.3. All cited applications are incorporated herein by reference for all purposes.

### Introduction

High-density oligonucleotide arrays (Chee, M., Yang, R. Hubbell, E., Berno, A. Huang, X. C., Stern, D., Winkler, J., Lockhart, D. J., Morris, M. S., Fodor, S.P.A 0.1996. Accessing Genetic Information with High-Density DNA Arrays. Science 274: 610-614; Lipshutz, R. J., Fodor, S. P., Gingeras, T. R., Lockhart, D. J. 1999. High density synthetic oligonucleotide arrays. Nature Genetics 21:20-24) have revolutionized the study of gene expression. The technology enables researchers to detect and quantify tens of thousands of transcripts in a single experiment, and has become a standard for the discovery of gene functions, drug evaluation, pathway dissection and classification of clinical samples (Lander, E. S. 1999. Array of hope. Nature Genetics 21:3-4). With the availability of the draft of the human genome sequence (Lander, E. S., Linton, L. M., Birren, B., Nusbaum, C., Zody, M. C., Baldwin, J., Devon, K., Dewar, K., Doyle, M., FitzHugh, W. 2001. Initial sequencing and analysis of the human genome. Nature 409: 860-921; Venter, J. C., Adams, M. D., Myers, E. W., Li, P. W., Mural, R. J., Sutton, G. G., Smith, H. O., Yandell, M., Evans, C. A., Holt, R. A. 2001. The sequence of the human genome. Science 291: 1304-1351), microarrays are capable of simultaneously monitor the whole expressed human genome. The expression profile of whole human genome will for the first time allow a detailed and comprehensive view of cellular processes, responses and their functional consequences.

### SUMMARY OF THE INVENTION

High-density oligonucleotide microarrays are standards for simultaneously monitoring expression levels of tens of thousands of transcripts. For accurate detection and quantitation of transcripts in the presence of cellular mRNA, it is desirable to design probes whose hybridization intensities accurately reflect the concentration of original mRNA. In one aspect of the invention, a model-based approach that predicts optimal probes using sequence and empirical information is employed for probe design (probe selection). Hybridization behavior can be described by a thermodynamic model, and the influence of empirical factors on the effective fitting parameters can be determined by modeling experimental data. According to the methods of the invention, multiple linear regression models can be built to predict hybridization intensities of each probe at given target concentrations and each intensity profile is summarized by a probe response metric. Probe sets are selected to represent each transcript, based on response and also independence (degree to which probe sequences are nonoverlapping), and uniqueness (lack of similarity to sequences in the expressed genomic background) using an optimization program. This approach is capable of selecting probes with high sensitivity and specificity for high-density oligonucleotide arrays in a large-scale and systematic manner.

### BRIEF DESCRIPTION OF THE FIGURES

The accompanying drawings, which are incorporated in and form a part of this specification, illustrate embodiments of the invention and, together with the description, serve to explain the embodiments of the invention:

FIG. 1. −ΔΔG values for the twenty-five probe base positions. Fitted weight coefficients, Wₓᵢ, for Equation 7 were generated by MLR analysis (Methods), given probe sequences for 90 HTC targets, and Ln(I) values for 8 pM target concentrations. The fitted weights are the effective −ΔΔGₓᵢ values for the bases: C (red curve),G (green curve), and T (yellow curve) in each sequence position, i, (i=1 to 25 from the 3′end of the probe), relative to the reference base, A, in the same position. (A) −ΔΔG values, given PM probe Ln(I) values (B) −ΔΔG values, given MM probe Ln(I) values.

FIG. 2. Predicted and the observed Ln(I) values, given 8 pM target. Profiles are for predicted (blue line) and observed (red dots) Ln(I) values for consecutive 25mer probes covering the base positions of the target sequence. Predicted Ln(I) values were computed using MLR solutions to Equation 7. (A) Ln(I_{PM}) values for yeast target, YCL055W. Training set consisted of hybridization intensities of PM probes covering 98 YTC targets (excluding YCL055W), spiked at 8 pM. (B) Ln(I_{MM}) for yeast target, YCL055W. Training set consisted of hybridization intensities of MM probes covering 98 YTC targets (excluding YCL055W), spiked at 8 pM. (C) Ln(I_{PM}) values for human target, X51688_{—}2844. Training set consisted of hybridization intensities of PM probes covering 89 HTC targets (excluding X51688_{—}2844), spiked at 8 pM. (D) Ln(I_{MM}) values for human target, X51688_{—}2844. Training set consisted of hybridization intensities of MM probes covering 89 HTC targets (excluding X51688_{—}2844), spiked at 8 pM

FIG. 3. Profiles of observed Ln(I) vs. Ln ([T]) values for six probes. The Ln(Kₐₚₚ) (intercept) values for the six probes range from 2.0 to 7.3 (shown in the label for each curve). The solid black line, is the best fit (least squares) line that relates Ln(I) to Ln ([T]) for the given probe.

FIG. 4. Relationships between average Ln-Ln slopes and average Ln(Kₐₚₚ) values for three model equations. Points for are average observed Ln-Ln slopes are red dots. Average predicted Ln-Ln slopes are shown for the Linear Model (blue squares) and Sigmoid Model (yellow triangles). Ln-Ln slope and Ln(Kₐₚₚ) values are the slope and intercept, respectively, of best fit (least squares) line for Ln(I) vs. Ln([T]), where [T] (pM) includes {0.25, 0.50, 0.75, 1.00, 1.50, 2.00, 3.00, 4.00, 6.00, 8.00, 12.00, 16.00}. Ln (I) values are either observed in the Latin square experiment, or predicted from concentration specific MLR model, for the given [T]. MLR training set data consisted of intensities and sequences for approximately 52,000 probes covering 90 HTC targets. Averages were computed for subsets of probes whose predicted Ln(Kₐₚₚ) values fell within each window (width 0.2) over the range Ln(Kₐₚₚ) values. (A) Ln-Ln slopes predicted by the First Approximation Linear model (Equation 7). (B) Ln-Ln slopes predicted by the Sigmoid Model (Equations 21, 22, Ceiling=8.5). (C) Ln-Ln slopes predicted by the Prediction Model. The Prediction Model used the Linear Model (Equation 23) to predict Ln-Ln slopes of probes (blue squares) whose predicted Ln(Kₐₚₚ) values fell below 5.7; and the Sigmoid Model to predict Ln-Ln slopes of probes (yellow triangles) whose predicted Ln(Kₐₚₚ) exceeded 5.7.

FIG. 5. Predicted (blue line) and observed (red dots) Ln-Ln slopes. Ln-Ln slopes are shown for probes covering a HTC target, AL049450_{—}1209. Predicted Ln-Ln slopes and Ln(Kₐₚₚ) values were computed as described in the legend to FIG. 4. MLR training sets consisted of Latin Square data 89 for HTC targets. Probes for AL049450_{—}1209 were not included in the training set. The arrow indicates a region consisting of high affinity probes. (A) Ln-Ln slopes predicted by the Linear Model alone. The correlation coefficient for predicted vs. observed Ln-Ln slopes is 0.69. (B) Ln-Ln Slopes predicted by the Prediction Model. The Prediction Model was implemented as described in the legend for FIG. 4. The correlation coefficient between prediction and observed Ln-Ln slopes is 0.83.

FIG. 6. Change call sensitivity increases with probe set score. Each point gives the sensitivity and average probe set score for 84 probe sets (11 probe pairs per set) for 84 HTC transcripts. Each point represents a separate probe set selection for the 84 genes, where a ceiling on the response values of candidate probes forced the selection of probe sets with less than optimal probe set scores. Sensitivity is equal to the fraction of the probe sets for which the Change call algorithm correctly calls an Increase change of target concentration from 1 pM to 2 pM in four replicate pairs of Latin Square experiment. Avg Probe Set Score is the average over 84 probes set scores for the point. The horizontal bars are standard deviations of the average probe set scores.

FIG. 7. Comparison of intensity property profiles. Probe sets are comprised of N=16 probe pairs, selected by the heuristic method (yellow diamonds); and N=11 (blue triangles) and N=16 (red squares) probe pairs, selected by the model-based method. Medians of probe set intensity property values are taken over 99 YTC probe sets for each target [T] concentration. Each probe set intensity property is the median of property (P) for the N selected probes. (A)P=Ln(I_{PM}). (B) P=intensity discrimination, ((I_{PM}−I_{MM})/(I_{PM}+I_{MM})).

FIG. 8. Comparison of Call Sensitivity Profiles. Probe sets are comprised of N=16 probe pairs, selected by the heuristic method (yellow diamonds); and N=11 (blue triangles) and N=16 (red squares) probe pairs, selected by the model-based method. N is the number of probe pairs in the probe set. Sensitivity and specificity values were determined for probe sets of 99 yeast genes in four replicate Latin square experiments (Rep). Sensitivity values are given for each base target [Tₜ] concentration. Algorithm parameters were adjusted to achieve the same specificity values for the three types of probe sets in a given comparison study. A. Comparison of Two-Fold Change Call Sensitivity. Sensitivity is equal to the fraction of Increase calls out of the total comparisons, where the target concentration is a two-fold increase relative to the base concentration (twofoldRepPair). The number of total comparisons is 1584 change calls (1 Change Call/twofoldRepPair×16 twofoldRepPairs/Gene×99 Genes). Specificity is equal to the average fraction of NoChange calls out the comparisons, where the target and base concentrations were the same. Algorithm parameters were adjusted to achieve specificities of 99.0%. B. Detection Call Sensitivity Profiles. Sensitivity is equal to the fraction of detection (Present) calls out a total of 396 calls (1 Detection Call/Rep×4 Reps/Gene×99 Genes), for the given concentration. Specificity is equal to the fraction not detection (Absent) absent when [T] equals zero. Algorithm parameters were adjusted to achieve specificities of 93.2%.

FIG. 9 Example of probe selection. Observed slopes of all 25mer probes are shown for one HTC target. Large Blue circles enclose the Ln-Ln slopes of eleven probes selected by new model-system, and small yellow circles enclose the Ln-Ln slopes of 16 probes selected by the previous heuristic system.

FIG. 10 Correlation Coefficients for Predicted vs. Observed Values for YTC (99 Yeast test array genes) and HTC (90 Human test array genes) Latin Square Datasets, Using the Prediction Model.

### Detailed Description of the Embodiments of the Invention

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

I. General

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. Nos. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com. The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. No. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. No. 6,361,947, 6,391,592 and U.S. patent application Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent application No. 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application No. 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. patent application Ser. No. 10/063,559, Nos. 60/349,546, 60/376,003, 60/394,574, 60/403,381.

II. Glossary

The following terms are intended to have the following general meanings as there used herein.

Nucleic acids according to the present invention may include any polymer or oligomer of pyrimidine and purine bases, preferably cytosine (C), thymine (T), and uracil (U), and adenine (A) and guanine (G), respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be deoxyribonucleic acid (DNA) or ribonucleic acid (RNA), or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA), which may be isolated from natural sources, recombinantly produced or artificially synthesized and mimetics thereof. A further example of a polynucleotide of the present invention may be peptide nucleic acid (PNA) in which the constituent bases are joined by peptides bonds rather than phosphodiester linkage, as described in Nielsen et al., Science 254:1497-1500 (1991), Nielsen Curr. Opin. Biotechnol., 10:71-75 (1999). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

Nucleic acid library or array is an intentionally created collection of nucleic acids which can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a column by m row matrix of the building blocks to be added. The switch matrix is all or a subset of the binary numbers, preferably ordered, between l and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer.

Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers. Initiation Biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Complementary or substantially complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid.” The proportion of the population of polynucleotides that forms stable hybrids is referred to herein as the “degree of hybridization”.

Hybridization conditions will typically include salt concentrations of less than about 1 M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the thermal melting point™ fro the specific sequence at s defined ionic strength and pH. The Tm is the temperature (under defined ionic strength, pH and nucleic acid composition) at which 50% of the probes complementary to the target sequence hybridize to the target sequence at equilibrium.

Typically, stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning A laboratory Manual” 2nd Ed. Cold Spring Harbor Press (1989) and Anderson “Nucleic Acid Hybridization” 1st Ed., BIOS Scientific Publishers Limited (1999), which are hereby incorporated by reference in its entirety for all purposes above.

Hybridization probes are nucleic acids (such as oligonucleotides) capable of binding in a base-specific manner to a complementary strand of nucleic acid. Such probes include peptide nucleic acids, as described in Nielsen et al., Science 254:1497-1500 (1991), Nielsen Curr. Opin. Biotechnol., 10:71-75 (1999) and other nucleic acid analogs and nucleic acid mimetics. See U.S. Pat. No. 6,156,501 filed Apr. 3, 1996.

Hybridizing specifically to: refers to the binding, duplexing, or hybridizing of a molecule substantially to or only to a particular nucleotide sequence or sequences under stringent conditions when that sequence is present in a complex mixture (e.g., total cellular) DNA or RNA.

Probe: A probe is a molecule that can be recognized by a particular target. In some embodiments, a probe can be surface immobilized. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

Effective amount refers to an amount sufficient to induce a desired result.

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, a cRNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

A fragment, segment, or DNA segment refers to a portion of a larger DNA polynucleotide or DNA. A polynucleotide, for example, can be broken up, or fragmented into, a plurality of segments. Various methods of fragmenting nucleic acid are well known in the art. These methods may be, for example, either chemical or physical in nature. Chemical fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3rd Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range. Useful size ranges may be from 100, 200, 400, 700 or 1000 to 500, 800, 1500, 2000, 4000 or 10,000 base pairs. However, larger size ranges such as 4000, 10,000 or 20,000 to 10,000, 20,000 or 500,000 base pairs may also be useful.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

Single nucleotide polymorphism (SNPs) are positions at which two alternative bases occur at appreciable frequency (>1%) in the human population, and are the most common type of human genetic variation. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

Genotyping refers to the determination of the genetic information an individual carries at one or more positions in the genome. For example, genotyping may comprise the determination of which allele or alleles an individual carries for a single SNP or the determination of which allele or alleles an individual carries for a plurality of SNPs. A genotype may be the identity of the alleles present in an individual at one or more polymorphic sites.

III. Oligonucleotide Probe Design

One technical challenge of representing the human genome on oligonucleotide microarrays is to select probes that can monitor the entire expressed genome in one or two microarrays. Quantitative detection of transcripts requires that probes exhibit a sensitive and predictable response to concentrations of the specific targets of the probes. This response must occur in the presence of a complex mixture of nonspecific targets. High density oligonucleotide probe array design typically uses multiple short (25mer) oligonucleotides probes to represent each transcript. One advantage of multiple probes is that they enable statistical assessment of expression measurements; the disadvantage is that they occupy space on the microarray. Thus it is desirable to select more optimal probes to represent each transcript.

In one aspect of the invention, a model-based approach for prediction of optimal probe sets is provided. In one example, custom high density oligonucleotide arrays that contained 25mer probe sequences to represent approximately two hundred yeast and human transcripts (the targets of the array) were used in a series of experiments which provide data for model building. The target transcripts were spiked into a mixture of labeled mRNA from human tissues (the genomic background) at variable concentrations. The data generated by this experimental system was used to model the relationship between hybridization intensities and ΔG_{d}, the free energy difference between a target-probe duplex, and the unbound target and probe. Analysis of effective fitting parameters indicates that the sequence positions of probe bases contribute to duplex stability. In addition, it was shown that the property of high hybridization intensity alone, does not ensure that a probe's intensity will vary in response to varying concentrations of its specific target.

In some embodiments, the probe selection system of the invention is therefore based on a prediction model for probe response, a metric that measures the intensity (I) response on the Ln-Ln (natural logarithm) scale to target concentration. The prediction model combines multiple linear regression (MLR) models that predict the Ln(I) values for a given target concentration. The probe selection system selects probe sets that are optimized with regard to this response metric and also uniqueness and independence. Our new method combines a formal thermodynamic model with empirically derived parameters to fundamentally change the method of designing expression arrays.

The Physical Model.

Duplex formation in the microarray system occurs between a probe with one end tethered to a surface and a target in solution. The target (T) hybridizes to its complementary probe (P) to form a target-probe duplex (T·P), and the reaction is accompanied by a favorable (negative) free energy change, ΔG_{d}, that measures the stability of the duplex. The stability of the duplex is influenced by stacking energies and by hydrogen bonding between target-probe base pairs. Experiments show that duplex stability appears to depend not only on the compositions of the base pairs, but also on the positions of the probe bases relative to the ends of the probe. Competing unfavorable interactions, such as probe self-folding, probe-to-probe interaction, target self-folding, and target-to-target interaction, can interfere with duplex stability.

In one aspect of the invention, models for the sequence dependence of ΔG_{d} was developed, which take into account the positional contributions of each base to duplex stability, and a subset of the possible unfavorable interactions.

Positional contributions were modeled as the sum of contributions to ΔG_{d}. from each base at each position. When contributions from all four bases are considered, the MLR model is over-specified and weight coefficients are poorly determined, because the presence or absence of the fourth base at a given position is determined by the other three. In order to avoid this, the A base is held as a reference, and the relative free energy change, ΔΔG_{d}, can be modeled using the approach introduced by Hacia et al. (Hacia, J. G, Sun, B., Hunt, N., Edgemon, K., Mosbrook, D., Robbins, C., Fodor, S. P. A., Tagle, D. A., Collins, F. S. 1998. Strategies for Mutational Analysis of the Large Multiexon ATM Gene Using High-Density Oligonucleotide Arrays. Genome Research 8: pp 1245-1258). ΔΔG_{d} is the sum of ΔΔGₓᵢ values for each base, x,=C, G, T, in each sequence position, i, relative to the reference base, A, in the same position,

$$ΔΔ {G}_{d} = {\sum }_{x = C , G , T}^{} {\sum }_{i = 1}^{N} ΔΔ {G}_{xi} {S}_{xi} \qquad [ 1 ]$$

where N is probe length and Sₓᵢ is the occupation variable,

$${S}_{xi} = \{ \begin{matrix} 1 , BaseInPosition , i = x \\ 0 , Otherwise\end{matrix} \qquad [ 2 ]$$

ΔΔG_{d} is offset from ΔG_{d} by a constant, c₀, the sum of ΔG_{Ai} values for the base, A, in each sequence position.

In some embodiments, three types of unfavorable interactions may be considerd; consecutive hairpins (ΔG_{HC}), non-consecutive hairpins (ΔG_{HN}); and G quartets, which are hydrogen-bonded G tetraplexes (Turner, D. H., 2000. Conformational Changes. In Nucleic Acids Structure, Properties, and Functions. (eds. Bloomfield, V. A., Crothers, D. M., Tinoco, I.) pp. 259-334. University Science Books, Sausalito, Calif.). The contribution to the free energy is considered separately for the presence of a G quartet in the beginning (ΔG_{b}), middle (ΔGₘ), and end (ΔGₑ) of the probe sequence. We combine terms for these unfavorable interactions with ΔΔG_{d} and express ΔG_{d} as

ΔG_{d}=ΔΔG_{d}+ΔG_{C}H_{C}+ΔG_{N}H_{N}+ΔG_{b}Q_{b}+ΔGₘQₘ+ΔGₑQₑ+C₀ [3]

where H_{N} and H_{C}, are variables for the potential of the probe sequence to form nonconsecutive and consecutive hairpins, respectively (Methods). The Gquartet variables, Q_{b}, Qₘ, and Qₑ, are counts of runs of four G bases in the beginning, middle, and end of the probe sequence (Methods). ΔG_{d} is related to [T·P], the concentration of the target-probe duplex.

$$[ T \cdot P ] = {C}^{*} {ⅇ}^{- Δ {G}_{d} / {RT}^{*} [ T ] [ P ]} \qquad [ 4 ]$$

The derivation and assumptions for Equation 4 are given in the Methods section. [T] and [P] are the total concentrations of target and probe, respectively, R is the Bolztman constant, T* is temperature, and C* is a constant.

### Prediction of Intensity Values

Based on the physical model, a linear equation for the sequence dependence of microarray intensity data can be derived, and used to build Multiple Linear Regression (MLR) models. Microarray data consists of fluorescent intensities (I) values (or other hybridization measurments), which are proportional to [T·P].

I=α[T·P] [5]

Use of Eqs. 4 and 5 gives,

Ln(I)=C₁+C₂ΔG_{d} [6]

for a given target concentration, [T], and where C₁(Ln(αC*[T][P])) and C₂ (1/(RT*) are constants. A linear equation, which relates Ln(I) to probe sequence terms, is derived by substituting Equation 3 into Equation 6, setting N=25 (for 25mer probes), replacing the products of all constants with a Weight (W) for the term, and summing all constants into a single constant term, W₀.

$$Ln ( I ) = {\sum }_{x = C , G , T}^{} {\sum }_{i = 1}^{25} {W}_{xi} {S}_{xi} + {W}_{C} {H}_{C} + {W}_{N} {H}_{N} + {W}_{b} {Q}_{b} + {W}_{m} {Q}_{m} + {W}_{e} {Q}_{e} + {W}_{0} \qquad [ 7 ]$$

Equation 7 serves as a first approximation model equation for MLR analysis (Methods).

In one example, the MLR was applied to intensity data generated from two custom high density oligonucleotide microarrays, yeast_test (YTC) and human-test (HTC) chips. These custom arrays contained all 25mer probes covering 600 to 1000-bp regions of 99 yeast and 90 human test chip transcripts; respectively. Two types of probe sequences covered each position in each transcript sequence; a perfect match (PM) probe with sequences exactly matching the cloned sequence, and a mismatch (MM) probe with a single substitution at the central position. Labeled cRNA targets were made for each clone, and spiked at known concentrations into pooled labeled human complex background (Methods). Intensity data sets were systematically collected over a 4000-fold concentration range according to a Latin square design (Methods).

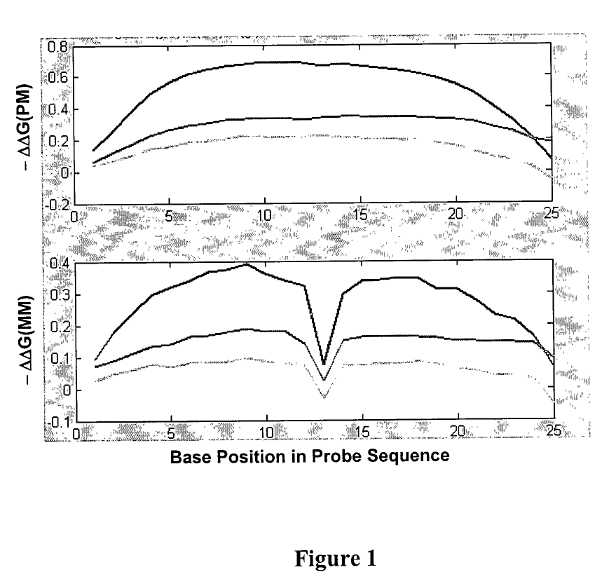

MLR analysis gives the fitted, weights Wₓᵢ, which are the effective −ΔΔGₓᵢ values for the contribution to duplex stability of each base, x, in each position, i. FIG. 1 shows profiles of these effective −ΔΔG values for C, G, and T bases at each base position in PM probes (FIG. 1A) and MM probes (FIG. 1B). The relative heights of the profiles show the relative contributions of the three bases at each position to ΔG_{d}. The height of the profile for the C base is higher than that of the other three bases, which is consistent with the higher stability of GC base pairs. The lower height of the G base profile, relative to the C base, might be due to the interference of labels on the C bases of target and other empirical factors. The ·ΔΔG values decrease at the 3′ and 5′ ends of the probe, suggesting that that bases at ends of the probe have decreased contributions to duplex stability. This is consistent with the cooperative behavior of duplex formation (Bloomfield, V. A., Crothers, D. M., Tinoco, 1. 2000. Nucleic Acids Structure, Properties, and Functions. (eds. Bloomfield, V. A., Crothers, D. M., Tinoco, I.), University Science Books, Sausalito, Calif.), and was also observed by Tobler et al. (Tobler, J. B., Molla, M. N., Nuwaysir, E. F., Green, R. D., and Shavlik, J. W. 2002. Evaluating machine learning approaches for aiding probe selection for gene-expression arrays. In BIOINFORMATICS Proceedings Tenth International Conference on Intelligent Systems for Molecular Biology. pp. S164-S171. Oxford University Press). When MLR analysis is applied to training set data consisting of MM probe intensities, the mismatch position in the center of the probe does not contribute to duplex stability as expected (FIG. 1B). In addition the −ΔΔG values for bases in positions flanking the central mismatch position are decreased. These observations suggest that the center position contributes significantly to duplex stability. Thus, the fitted weights produced by MLR solution to Equation 7 appears to model expected hybridization behavior.

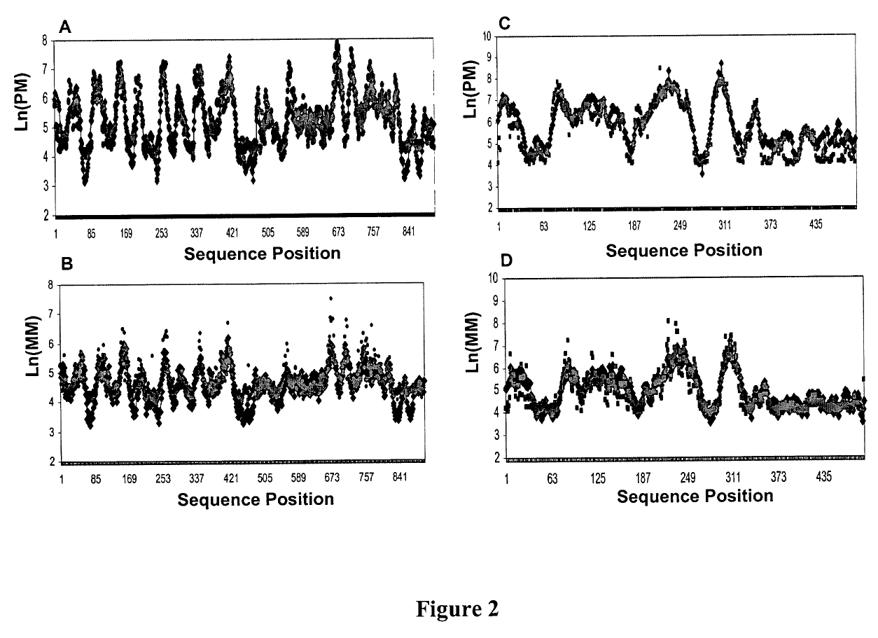

When MLR solutions to Equation 7 are used to predict Ln(I) values, there is good correlation with observed Ln(I) values. Profiles of Ln(I) values for consecutive probes that cover a target sequence are shown for a representative yeast (FIGS. 2A-B) and human (FIGS. 2C-D) target spiked at 8 pM. The correlation coefficients (0.85 and 0.90 for yeast and human targets, respectively) for predicted vs. observed Ln(I_{PM}) values are higher than the correlation coefficients(0.76 and 0.88 for yeast and human respectively) for the Ln(I_{MM}) values, as expected. The good correlation coefficients hold for a 4000-fold target concentration range (Data not shown).

### Probe Response Metric

The ability to predict Ln (I) values from probe sequence provides a foundation for probe selection. One essential criterion of probe selection for a quantitative expression analysis is that hybridization intensities of the selected probes have a predictable response to target concentrations, [T].

Equation 6 may be rewritten as

Ln(I)=Ln(Kₐₚₚ)+Ln([T]) [8]

where Kₐₚₚ (the apparent affinity constant)=αC*e^{-ΔG}^{_{d}}^{/RT} [P]. However, the derivation of equation 6 is based on a number of simplifying assumptions (see Methods and Discussion), especially [T·P]<<[P], that is the fraction of occupied probe sites in a probe feature (a particular area on the array, covered by a set of probes with a common sequence) is always negligible compared to the total number of available sites. In fact it is a feature of microarray hybridization behavior that probe sites may approach chemical saturation (all probe sites are occupied) due to high specific target concentrations, and/or due to high probe hybridization affinities.

Empirical adjustments to the first approximation equations may be made so that the models produce a better fit to the observed data. Specifically, it was observed that the data better fits the form

Ln(I)=Ln(Kₐₚₚ)+SLn([T]) [9]

where 0<S<1. This is primarily due to the onset of chemical saturation of the probe feature.

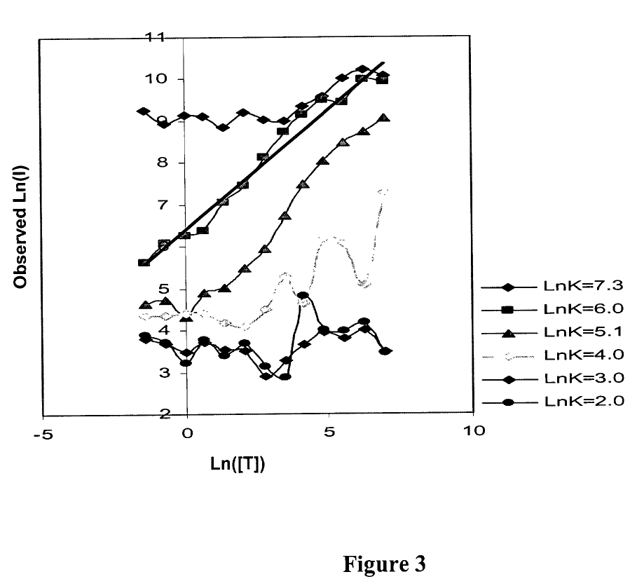

Ln(Kₐₚₚ) is the intercept and S is the slope (the Ln-Ln slope) of the line that relates Ln(I) to Ln([T]) (black line, FIG. 3). As the Ln-Ln slope approaches one, the relationship between I and [T] approaches the ideal linear form, I=Kₐₚₚ[T]. Selection of probes with maximal Ln-Ln slopes maximizes the degree and the linearity of the intensity response to target concentration. Therefore, in some embodiments, the Ln-Ln slope is set to be the probe response metric.

Ln-Ln slopes are computed by building MLR models for each target concentration [T] in the Latin square data. Then for a given probe sequence, Ln(I) values are predicted for each [T] (0.25-1024 pM), using the set of concentration specific MLR models, and the Ln-Ln slope is the slope of the best fit (least squares) line that relates Ln(I) to Ln([T]). The range of [T] used for the probe response metric was 0.25 (note that 0.25 pM is less than one copy per cell) to 16 pM, because Expression Detection performance was improved for probe sets whose selections used this range.

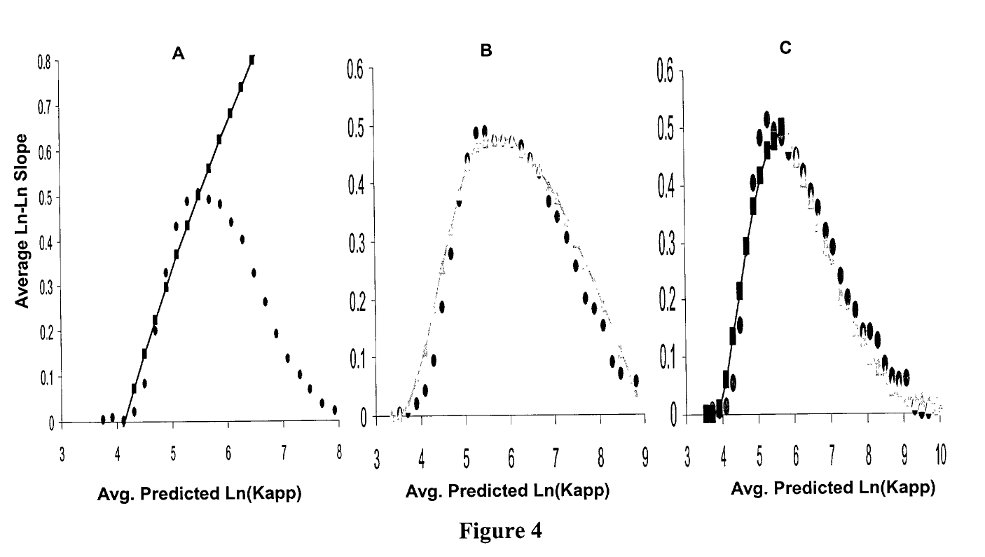

The relationship between Ln-Ln slope and Ln(Kₐₚₚ) shows that there two classes of unresponsive probes: probes with very high and probes with very low hybridization affinities. Probes with low Ln(Kₐₚₚ) values also have low Ln-Ln slopes. Such probes (FIG. 3, brown and green). are unresponsive to target concentration due to low hybridization affinities. As Ln(Kₐₚₚ) increases, Ln-Ln slopes increase (FIG. 3, pink and red) and probes are responsive. However, probes, whose Ln(Kₐₚₚ) values exceed a threshold, exhibit decreasing Ln-Ln slopes with increasing Ln(Kₐₚₚ) values (FIG. 3, blue, and FIG. 4, red dots). These high affinity probes are increasingly unresponsive to specific target because they by cross-hybridizing to nonspecific targets in the complex genomic background, and saturate their binding sites.

### Additional Model

As discussed above, the first approximation equation does not assume that probe features may approach chemical saturation. FIG. 4A shows that the first approximation (Equation 7) makes Ln-Ln slope predictions that track well with observed Ln-Ln slopes for probes whose Ln(Kₐₚₚ) values fall below the threshold. The threshold is the Ln(Kₐₚₚ) value that produces the maximum observed Ln-Ln slope. However this model cannot predict the decreasing response of increasingly high affinity probes above the threshold. Instead it predicts that Ln-Ln slopes continue to increase, which is the behavior that would be observed in the absence of chemical saturation. It was observed that a sigmoid equation, which incorporates the existence of a Ceiling for Ln(I) values that is approached due to chemical saturation, gives a better fit than the linear equation (7). We also find that addition of interaction terms,

$${\sum }_{j = A , C , G , T}^{} {W}_{j} {B}_{j}^{2} ,$$

where B=Number of Basetype j, improves the model. Inclusion of the interactions terms and use of a sigmoid function to relate Ln(I) to ΔG_{d} gives the sigmoid model (Equations 21 and 22, Methods). As shown in FIG. 4B, the sigmoid model predicts that Ln-Ln slopes decrease when Ln(Kₐₚₚ) values exceeds a threshold.

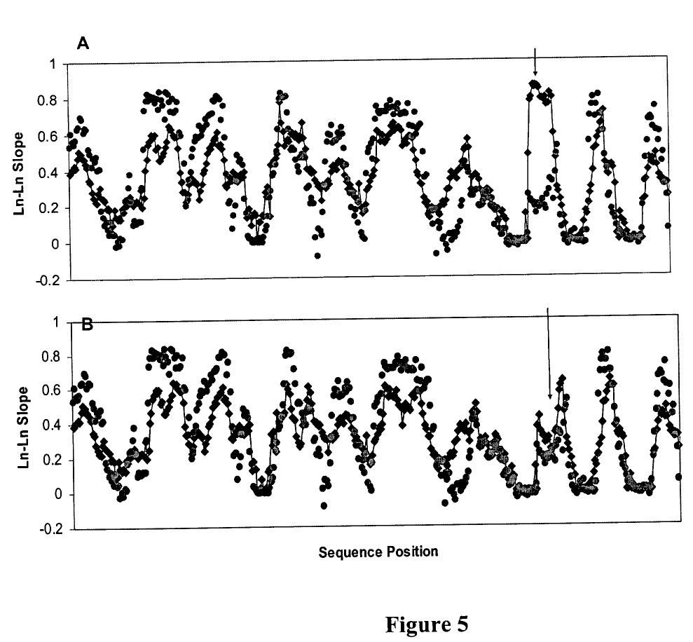

Although the sigmoid model is capable of making accurate slope predictions throughout the entire Ln(Kₐₚₚ) range, it was found that the linear model gives more accurate and generalizable predictions for probes with Ln(Kₐₚₚ) below the threshold. The linear model is a version of the first approximation model that includes the interaction terms (Equation 23, Methods). Thus, a prediction model (FIG. 4C) was created. The prediction model combines the linear and sigmoid models by using the sigmoid model for probes whose predicted Ln(Kₐₚₚ) values exceed a threshold, and the linear model for probes whose predicted Ln(Kₐₚₚ) values fall below a threshold. FIG. 5 gives an example of the improved performance that results from using the prediction model (FIG. 5B) relative to the linear model alone (FIG. 5A). The combined prediction model attenuates the over-prediction of high affinity probes (arrow, FIG. 5).

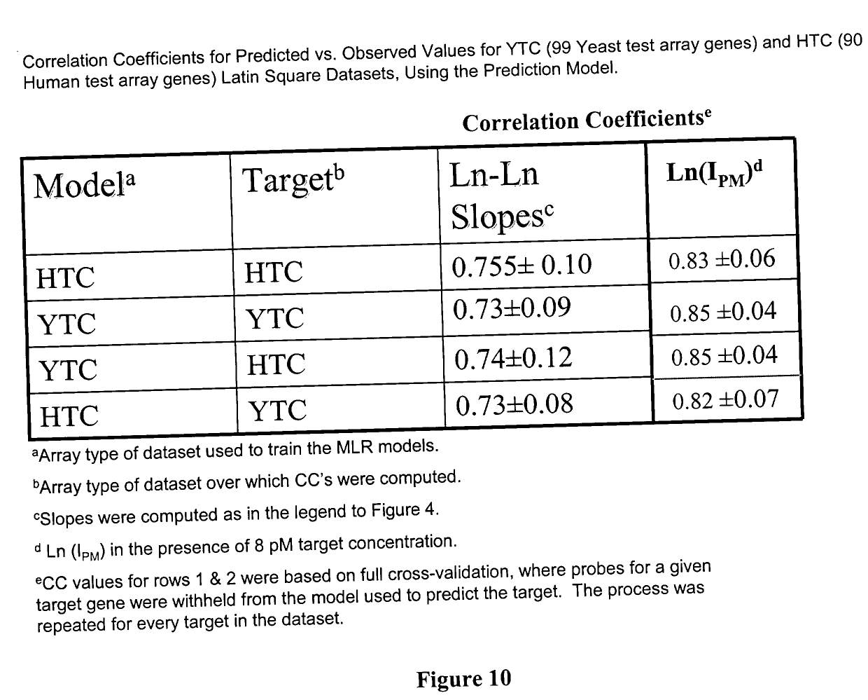

FIG. 10 summarizes average correlation coefficients between predicted and observed values, using the prediction model, and data for 90 HTC and 99 YTC transcripts. Average correlation coefficients are broken out according to the array type of the data used to train the model, and the array type of the data predicted by the model. Full cross-validation was employed for cases in which the dataset used for training the models was the same as the dataset used as the target of the model (rows one and two). Average correlation coefficients for the four rows in FIG. 10 are around 0.84 for Ln(I_{PM}) values, and 0.74 for slope values. Slope values are predicted with lower correlation because the fit is through a set of predicted Ln(I_{PM}) points and covers the lower range (0.25-16 pM) of target concentrations. The prediction model appears to generalize well because models trained on YTC data can be used to predict HTC data, and vice-versa (rows three and four).

### Probe Selection System

To generate an optimal probe set for each transcript, it is also desirable to consider two other metrics: uniqueness and independence. Uniqueness, U, identifies whether a probe is likely to cross-hybridize to other known expressed sequences in the genomic background. U is either zero (not unique) or one (unique) based on a sequence similarity rule. The rule was derived based on a study that employed custom cross-hyb microarrays that included 794 different perfect match probes and 333 specific mismatches. Yeast transcripts were spiked into a complex hybridization mixture according to the yeast test chip Latin square design and hybridized to the cross-hyb arrays. Multiple cross-hybridization rules were used to determine which one best differentiated between probes which show significant cross-hybridization to the mismatch probes and those which do not. Based on this a probe is considered to be unique if it does not have at least two 8mer perfect matches, including at least twelve consecutive bases matching bases, to any other sequences in the expressed genomic background. This rule was tested against a different custom array which had 2694 probes with 100 random mismatch probes to each perfect match probe.

Independence defines the degree to which regions in the target sequence that are selected for complementary probe synthesis, are well separated or non-overlapping. In general, we expect probes whose sequences overlap to be vulnerable to similar systematic errors, such as cross hybridization, synthesis efficiency, and secondary structure. Therefore, all else being equal, a set of eleven 25mer probe sequences, selected from 35 bases of target sequence with an overlap of 24/25 bases for each probe, is much less desirable than a set of eleven probe sequences, selected from 275 bases of target sequence with no overlap. We therefore introduce a penalty term, D, based on the distance between the positions, P, in the target sequence that align with the centers of two consecutive probes, i, and i+1.

D_{i,i+1}=max(1, {square root over ((Pᵢ−Pᵢ₊₁)/R))}). [10]

The form of D meets several criteria for a multiplicative distance penalty. First, we expect probes overlapping by 24/25 bases to be undesirable, but not completely useless, and therefore the penalty should always be positive. Second, the penalty of overlap should decrease smoothly as we increase the center-center distance. Third, there are theoretical reasons for believing that the covariance between two overlapping probes will follow the square root of the overlap. Finally, increasing the distance after some range, R, should add no additional benefit. R is chosen to be fifteen based on empirical fits.

The model-based probe selection is implemented by a system takes the transcript sequence as input, and uses a dynamic program to select a probe set that optimizes a probe set score. The equation for probe set score combines response, uniqueness, and distance penalty metrics are into a single value for N probes.

$$Probe set Score = {\sum }_{i = 1}^{n} {S}_{i} {U}_{i} {D}_{i , i + 1} \qquad [ 11 ]$$

MM probes are generated from the PM probe sequences. The system has been used for large-scale probe selections of whole organism expressed genomes, including the Hg_U133 human genome GeneChip® microarrays.

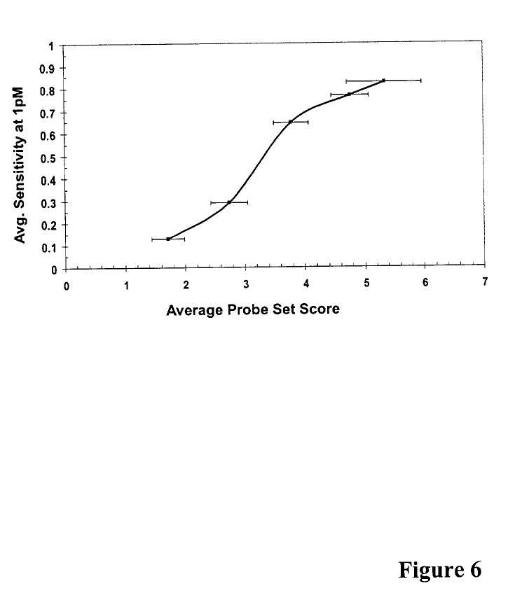

The performance of probe sets, selected by the dynamic program, was compared to probe sets deliberately selected to be less than optimal with regard to probe set score. FIG. 6 shows the relationship between one performance metric, Change call sensitivity (described below) and average probe set score. Performance increases with average probe set score up to the optimal (last point) score, indicating that probe set score is a good continuous metric to use in the search for the optimal probe sets.

Probe Set Performance

In this section we compare performance of probe sets, selected by the heuristics rules, to that of probe sets, selected by the model-based system. Performance metrics include profiles of probe set intensities and sensitivities achieved by expression Call algorithms (Detection and Change). The expression call algorithms use a set of N probe pairs, to choose between alternative calls in a statistical test (Liu et al. 2002). The Detection Call indicates whether a transcript is detected (Present) or not detected (Absent). The Change Call (Increase, Decrease, and No Change) indicates whether or not a transcript in one experiment is expressed at a different level in a second experiment.

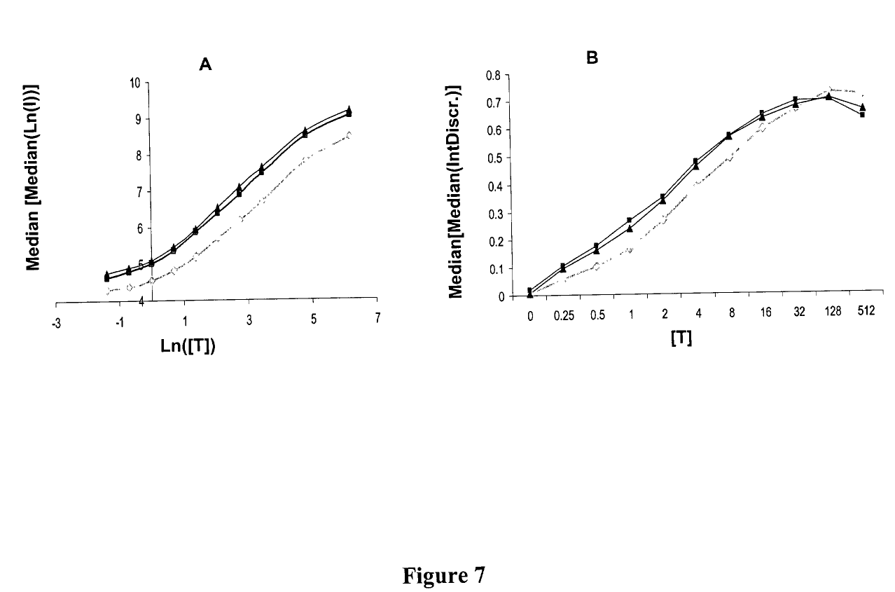

FIG. 7A compares median Ln(I) values over sets of eleven and sixteen PM probes, selected by the model-based method, to that of sixteen probes selected by the heuristic method. Both methods select well-behaved probes sets with median Ln (I_{PM}) values that respond well to increasing Ln ([T]) values. The model based system appears to select more optimal probe sets in that overall Ln(I_{PM}) values are increased. These PM intensities increase relative to MM intensities as shown by the increase in median intensity discrimination (I_{PM}−I_{MM}))/(I_{PM}+I_{MM})) profiles, given model-based probe selection (FIG. 7B). Intensity discrimination increases overall, except when no target is present, at which point intensity discrimination correctly approximates zero.

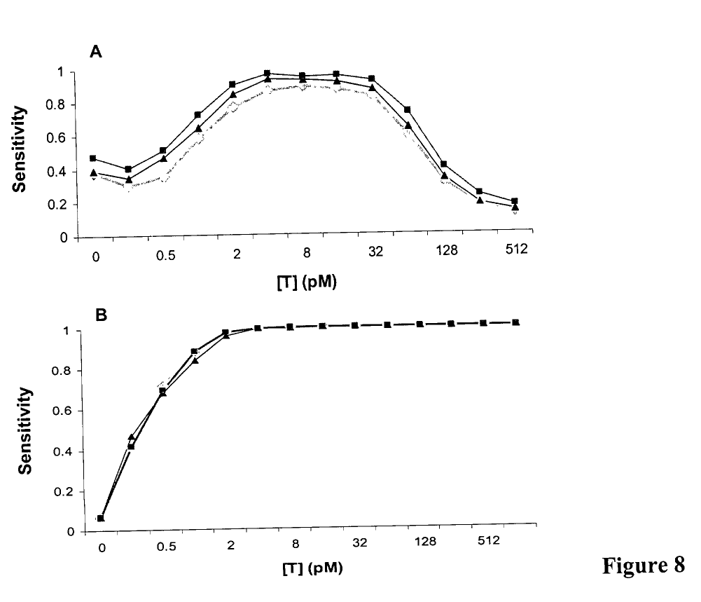

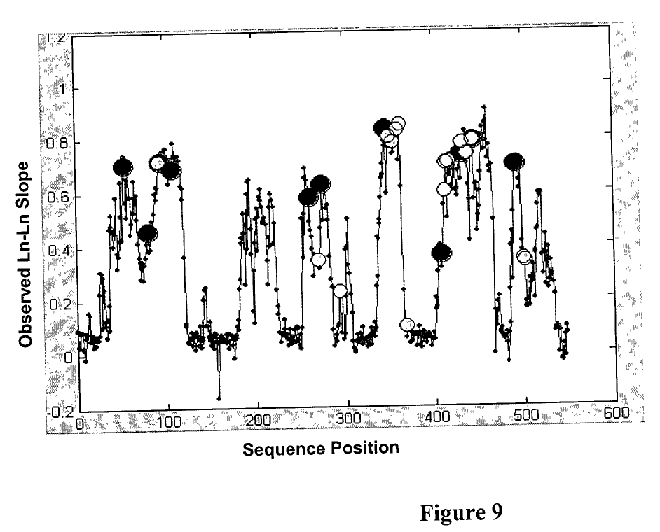

Expression calling performance serves as a functional test to indicate whether the model based probe selection system is capable of identifying probes with high sensitivity and specificity. FIG. 8 compares sensitivities of sets of eleven and sixteen PM probes, selected by the model-based method, to that of sixteen probes selected by the heuristic method. Algorithm parameters were adjusted to achieve the same specificity values for the three types of probe sets. FIG. 8A shows that sensitivity increases for change calls, given model-based probe selection and probe sets of size eleven as well as sixteen. For detection calls, the three selections performed equivalently (FIG. 8B), despite improvement in PM intensity, intensity discrimination, and change calls sensitivities, given the model-based based probe sets (FIG. 7 and FIG. 8A). It appears that detection calls were not sensitive to apparent intensity improvements. However, probe sets of size 11 appear to be sufficient to maintain detection call sensitivity, despite the loss of statistical power due to decreasing sample size. Similar results are achieved by probe sets for human test chip data (data not shown). FIG. 9 gives a representative example of probes selected by the heuristic and model-based systems. The new models and selection criteria achieve the goals of selecting probes with higher Ln-Ln slopes on average, than those selected by the previous heuristic system, and also achieves better spacing between probes for more independent sampling of the target.

### III. EXAMPLE.

The example demonstrates the ability to model microarray hybridization intensities and build a prediction model that captures the sequence dependence of the complex hybridization behavior of immobilized probes in the presence of whole genomic backgrounds. The prediction model generates a continuous and quantitative metric for probe response. The combination of this response metric along with uniqueness and independence criterions enables selection of optimal probe sets in a systematic and large-scale manner. The system provides the potential to reduce the size of probe sets on the high density oligonucleotide expression arrays from sixteen to eleven, while maintaining high sensitivity and specificity.

Methods

Latin Square Experiments

Yeast and human cRNA transcripts (the targets) were spiked into labeled complex human backgrounds at known concentrations and hybridization intensities were obtained for yeast-test chips (YTC) and human-test chips (HTC). Target groups were arranged in a classic Latin square design (Box et al. 1978) so that each hybridization mixture contained at least one target at each chosen target [T] concentration. Ninety-nine yeast cRNA targets for YTC experiments were spiked at fourteen concentrations ranging from 0.25 to 1024 pM in two-fold dilution steps, and included zero pM(no target present). Ninety cRNA targets for HTC experiments were spiked at sixteen pM concentrations that included, 0.0, 0.25, 0.50, 0.75, 1.00, 1.50,2.00, 3.00, 4.00, 6.00, 8.00, 12.00, 16.00, 32.00, 128.00, and 512. HTC targets included six bacterial and eighty-four human cRNAs. The complex background for YTC experiments consisted of labeled mRNA from four human tissues: fetal brain, liver, lung and testis. The complex background HTC experiments consisted of labeled mRNA from heart tissue where the target genes were knockout in vitro. Hybridization intensities were generated for the experiments according to the standard procedures for GeneChip® expression arrays.

Multiple Linear Regression (MLR)

Multiple linear regression was implemented by the function Regress (MathWorks 2000) to fit the weight coefficients of equations, 7,22, or 23. Values for dependent variables, Ln(I) or Y (Eq. 20), were computed from the intensities of hybridized targets, spiked according to the Latin square design (above). Each training set consisted of a subset Latin square hybridization intensities produced by targets spiked at a common concentration. Values for independent variables were derived from probe sequences on the yeast and human test chips. H_{C} and H_{N}, are counts of the longest runs of consecutive and non-consecutive basepairs in hairpin structures. Q_{b} Qₙ, Qₑ, count the number of runs of four G bases in regions: b=1-7, m=8-15, and e=16-22. Other independent variables are described in the Result section.

MLR models were evaluated by the correlation coefficients for predicted vs observed values. All such correlation coefficients were generated using the standard cross-validation method (Hastie et al. 2001), where test cases are held out of the cases used to train the model.

Derivations

[T·P] as a function of ΔG_{d}.

Assuming that

[T·P]<<[P] and [T·P]<<[T] [12]

where [T·P] is the surface concentration of target-probe duplexes. [P] is the total surface concentration of a feature, a set of probes with a common sequence covering a particular area on the array. [T] is the total concentration of intended target for a feature. Applying a first order kinetic model, we write the rate that target molecules bind to probe in terms of the rates of adsorption, rₐ and desorption, r_{d} as

d[T·P]/dt=rₐ−r_{d}. [13]

We assume rd depends only on [T·P]

r_{d}=k_{d}[T·P] [14]

and that rₐ depends on the concentration of unbound target, [Tᵤ], and unbound probe, [Pᵤ]We use Eq. (12) and assume [Pᵤ]≅[P], that is the fraction of occupied probe sites is negligible compared to the total number of available sites, and also assume [Tᵤ]≅[T]. Note that assuming [Pᵤ]≅[P] is a more serious assumption than assuming [Tᵤ]≅[T], because [T] is a bulk or volume concentration, while [P] is a surface concentration. Based on these assumptions

rₐ=kₐ[T]≅[P]. [15]

Assuming the reaction reaches equilibrium, d[T·P]/dt=0, we find

[T·P]=kₐk_{d}⁻¹[T][P]. [16]

We assume that target-probe duplex formation/dissociation is an on-off process (ie. we neglect nucleation and nucleotide zipper effects). Thus, we have a two-state population of completely bound or unbound target molecules in our model. It has been found that k_{α} has a relatively weak dependence on temperature and hence sequence of the probe from experiments on duplex formation of oligonucleotides in solution. One source of the modest sequence dependence is the nucleation barrier that should be sensitive to approximately five base pairs on the 5′ side of the probe (Bloomfield et. al. 2000). We will neglect nucleation effects and assume that k_{α} does not depend on sequence.

In sharp contrast, the desorption rate, k_{d}, can vary by many orders of magnitude depending on the sequence of DNA and is very sensitive to temperature (Bloomfield et. al. 2000). This is expected theoretically from reaction rate theory (Hanggi et al. 1990) where regardless of the dynamical regime that a system of reacting molecules is found (i.e. over-damped, under-damped) one finds a Van't Hoff-Arrhenius form for the desorption rate

$${k}_{d} = {k}_{d}^{0} {ⅇ}^{- Δ {G}_{de} / {RT}^{*}} \qquad [ 17 ]$$

where ΔG_{de} is the desorption activation free energy, T* is temperature, R is the Boltzman constant, and k_{d}⁰ is a molecular relaxation rate which depends on the shape of the potential, viscosity of the medium etc. Eq.(17) is both experimentally and theoretically well established for the case of simple molecules which react where ΔG_{de}/RT* >>1 (i.e. the condition of weak thermal noise). It has been shown that for short oligonucleotides in solution, this “on-off” model and the Arrhenius form give a reasonable description of the equilibrium population of bound and unbound molecules (Bloomfield et al. 2000). We assume that the adsorption activation free energy is negligible, so ΔG_{de}≅−ΔG_{d}, the free energy change for duplex formation Use of this assumption and Eq (16) and (17) give

$$[ T \cdot P ] = {C}^{*} {ⅇ}^{- Δ {G}_{d} / {RT}^{*}} [ T ] [ P ] \qquad [ 18 ]$$

where C*=kₐ/k_{d}⁰ is a constant independent of sequence

Linear and Sigmoid Model Equations

Using a sigmoid equation to relate Ln(I) (for a given target concentration) to ΔG_{d} gives

Ln(I)=Ceiling/(1+(Ceiling/N₀−1)e^{-rΔG}^{_{d}}) [19]

where N₀, r, and Ceiling are constants of the sigmoid equation. Taking the Ln of both sides and rearranging gives a linear form of the sigmoid equation

Y=C₃ΔG_{d}+C₄ [20]

where

Y=Ln((Ceiling−_{LnI})/_{LnI}) [21]

and C₃=−r; and C₄=Ln((Ceiling−N₀/N₀)) are constants.

We add four terms to Equation 3 (for ΔG_{d}):

$${\sum }_{j = A , C , G , T}^{} {W}_{j} {B}_{j}^{2} ,$$

where B=Number of Basetype j, and substitute the expanded Equation 3 into the Equation 20 to obtain the Sigmoid Model Equation

$$Y = {\sum }_{x = C , G , T}^{} {\sum }_{i = 1}^{25} {W}_{xi} {S}_{xi} + {W}_{C} {H}_{C} + {W}_{N} {H}_{N} + {W}_{b} {Q}_{b} + {W}_{m} {Q}_{m} + {W}_{e} {Q}_{e} + {\sum }_{j = A , C , G , T}^{} {W}_{j} {B}_{j}^{2} + {W}_{0} \qquad [ 22 ]$$

We substitute the expanded Equation 3 into Equation 6 to obtain the Linear Model Equation.

$$Ln ( I ) = {\sum }_{x = C , G , T}^{} {\sum }_{i = 1}^{25} {W}_{xi} {S}_{xi} + {W}_{C} {H}_{C} + {W}_{N} {H}_{N} + {W}_{b} {Q}_{b} + {W}_{m} {Q}_{m} + {W}_{e} {Q}_{e} + {\sum }_{j = A , C , G , T}^{} {W}_{j} {B}_{j}^{2} + {W}_{0} \qquad [ 23 ]$$

Results of the experiments are presented throughout the disclosure. The results showed that the prediction model produced good correlation coefficients for predicted vs. observed values, including Ln (I) and Ln-Ln slopes. It is sometimes desirable to have two different model equations, the sigmoid model equation (22) for high affinity probes and the linear equation (23) for lower affinity probes. The sigmoid model captures the nonlinear relationship between Ln(I) and ΔG_{d} and assumes that Ln(I) values will approach a Ceiling. This ceiling is more likely to be approached by high affinity probe sequences, because a finite amount of probe sequences on the solid support becomes chemically saturated with a mixture of specific and nonspecific targets. The sigmoid model was found to less accurate than the linear model, for lower affinity probes. This may be due to the assumption of a single Ceiling value required by the sigmoid model. However, the linear model results in over-prediction of the Ln-Ln slopes of high affinity probes in some case. Thus the best prediction results were achieved by combining the two models.

It is to be understood that the above description is intended to be illustrative and not restrictive. Many variations of the invention will be apparent to those of skill in the art upon reviewing the above description. The scope of the invention should be determined with reference to the appended claims, along with the full scope of equivalents to which such claims are entitled. All cited references, including patent and non-patent literature, are incorporated herewith by reference in their entireties for all purposes.

What is claimed is:

1. A method for predicting slope of the response of an oligonucleotide probe to its target comprising applying a sigmoid model for probe whose predicted Ln(Kₐₚₚ) values exceed a threshold, and a linear model for probes whose predicted Ln(Kₐₚₚ) values fall below a threshold;

2. The method of claim 1 wherein the linear model is
$$Ln ( I ) = {\sum }_{x = C , G , T}^{} {\sum }_{i = 1}^{25} {W}_{xi} {S}_{xi} + {W}_{C} {H}_{C} + {W}_{N} {H}_{N} + {W}_{b} {Q}_{b} + {W}_{m} {Q}_{m} + {W}_{e} {Q}_{e} + {\sum }_{j = A , C , G , T}^{} {W}_{j} {B}_{j}^{2} + {W}_{0}$$

3. The method of claim 2 wherien the sigmoid model is defined by:
Y=Ln((Ceiling−LnI)/LnI) and
$$Y = {\sum }_{x = C , G , T}^{} {\sum }_{i = 1}^{25} {W}_{xi} {S}_{xi} + {W}_{C} {H}_{C} + {W}_{N} {H}_{N} + {W}_{b} {Q}_{b} + {W}_{m} {Q}_{m} + {W}_{e} {Q}_{e} + {\sum }_{j = A , C , G , T}^{} {W}_{j} {B}_{j}^{2} + {W}_{0}$$

4. The method of claim 3 wherein the threshold is empirically determined.

5. The method of claim 5 wherein the threshold is approximately Ln(Kapp)=6.

6. A method for selecting oligonucleotide probes for gene expression monitoring comprising:
Comparing a candidate probe with sequences, other than its target, in the genomic background; and
Identifying the candidate as non-unique if it has a 16mer perfect matches or a 8mer and 12mer perfect matches, to any other sequences in the expressed genomic background.
